(19) United States
(12) Patent Application Publication
(10) Pub. No.: US 2004/0034040 A1
(43) Pub. Date: Feb. 19, 2004

(54) Use of thienopyrimidines

(76) Inventors: Hans-Michael Eggenweiler, Eggenweiler (DE); Volker Eiermann, Rodermark (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/432,778

(22) PCT Filed: Oct. 29, 2001
(86) PCT No.: PCT/EP01/12494

(30) Foreign Application Priority Data
Nov. 25, 2000 (DE) 100 58 663.5

(51) Int. Cl. A61K 31/519

(52) U.S. Cl. 514/260.1; 544/280

(57) ABSTRACT

The invention relates to the use of thienopyrimidines of formula (I)—wherein R¹, R², R³, R⁴ and X have the designations cited in claim 1—and the physiologically acceptable salts thereof, for producing a medicament for the treatment of angina, hypertension, pulmonary hypertension, congestive heart failure, arteriosclerosis, conditions of reduced patency of the heart vessels, peripheral vascular diseases, apoplexy, bronchitis, allergic asthma, chronic asthma, allergic rhinitis, glaucoma, irritable bowel syndrome, tumours, kidney failure, and cirrhosis of the liver, and for the treatment of female sexual disorders.

The invention relates to the use of compounds of the formula I

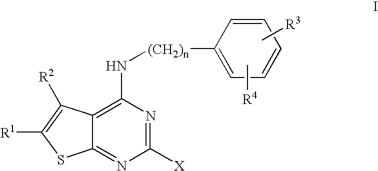

in which

R¹ and R² are each, independently of one another, H, A or Hal, where one of the radicals R¹ or R² is always ≠H,

R¹ and R² together are alternatively alkylene having 3-5 carbon atoms,

R³ and R⁴ are each, independently of one another, H, A, OA, OH or Hal,

R³ and R⁴ together are alternatively alkylene having 3-5 carbon atoms, —O—CH₂—CH₂—, —O—CH₂—O— or —O—CH₂—CH₂—O—,

X is R⁵ or R⁶ which is monosubstituted by R⁷,

R⁵ is linear or branched alkylene having 1-10 carbon atoms, in which one or two CH₂ groups may be replaced by —CH═CH— groups, or is —C₆H₄—(CH₂)ₘ—,

R⁶ is cycloalkylalkylene having 6-12 carbon atoms,

R⁷ is COOH, COOA, CONH₂, CONHA, CON(A)₂ or CN,

A is alkyl having from 1 to 6 carbon atoms,

Hal is F, Cl, Br or I,

m is 1 or 2, and

n is 0, 1, 2 or 3,

and their physiologically acceptable salts and/or solvates, for the preparation of a medicament for the treatment of angina, high blood pressure, high pulmonary pressure, congestive heart failure, atherosclerosis, conditions of reduced patency of heart vessels, peripheral vascular diseases, strokes, bronchitis, allergic asthma, chronic asthma, allergic rhinitis, glaucoma, irritable bowel syndrome, tumours, renal insufficiency, liver cirrhosis and for the treatment of female sexual disorders.

Pyrimidine derivatives are disclosed, for example, in EP 201 188 or WO 93/06104.

The use of other PDE-V inhibitors is described, for example, in WO 94/28902.

The invention had the object of finding novel compounds having valuable properties, in particular those which can used for the preparation of medicaments.

It has been found that the compounds of the formula I and their salts have very valuable pharmacological properties and are well tolerated.

In particular, they exhibit specific inhibition of cGMP phosphodiesterase (PDE V).

Quinazolines having a cGMP phosphodiesterase-inhibiting activity are described, for example, in J. Med. Chem. 36, 3765 (1993) and ibid. 37, 2106(1994).

The biological activity of the compounds of the formula I can be determined by methods as described, for example, in WO 93/06104. The affinity of the compounds according to the invention for cGMP and cAMP phosphodiesterase is determined by measuring their IC₅₀ values (concentration of the inhibitor needed to achieve 50% inhibition of the enzyme activity).

The determinations can be carried out using enzymes isolated by known methods (for example W. J. Thompson et al., Biochem. 1971, 10, 311). The experiment can be carried out using a modified batch method of W. J. Thompson and M. M. Appleman (Biochem. 1979, 18, 5228).

The compounds are therefore suitable for the treatment of illnesses of the cardiovascular system, in particular cardiac insufficiency, and for the treatment and/or therapy of impotence (erectile dysfunction).

The use of substituted pyrazolopyrimidinones for the treatment of female sexual disorders is described, for example, in WO 94/28902.

The compounds are effective as inhibitors of phenylephrine-induced contractions in corpus cavernosum preparations of rabbits. This biological action can be demonstrated, for example, by the method described by F. Holmquist et al. in J. Urol., 150, 1310-1315 (1993).

The inhibition of the contraction demonstrates the effectiveness of the compounds according to the invention for the therapy and/or treatment of impotence.

The invention relates to the use of the compounds of the formula I and their physiologically acceptable salts and/or solvates for the preparation of a medicament for the treatment of angina, high blood pressure, high pulmonary pressure, congestive heart failure, atherosclerosis, conditions of reduced patency of heart vessels, peripheral vascular diseases, strokes, bronchitis, allergic asthma, chronic asthma, allergic rhinitis, glaucoma, irritable bowel syndrome, tumours, renal insufficiency, liver cirrhosis and for the treatment of female sexual disorders.

The invention relates, in particular, to the use of the compounds of the formula I and their physiologically acceptable salts and/or solvates for the preparation of a medicament for the treatment of high pulmonary pressure.

The invention preferably relates to the use of 5-[4-(3-chloro-4-methoxybenzylamino)-5,6,7,8-tetrahydro-[1]-benzothieno-[2, 3-d]-pyrimidin-2-yl]-valeric acid and physiologically acceptable salts and/or solvates thereof for the preparation of a medicament for the treatment of high pulmonary pressure. The ethanolamine salt or the sodium salt is particularly preferred.

The compounds of the formula I can be employed as medicament active ingredients in human and veterinary medicine. They can furthermore be employed as intermediates for the preparation of further medicament active ingredients.

The invention accordingly relates to the compounds of the formula I and to a process for the preparation of compounds of the formula I according to claim 1 and their salts, characterised in that

a) a compound of the formula II

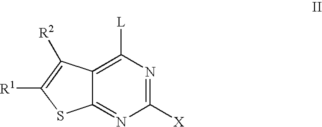

in which

R¹, R² and X are as defined above,

and L is Cl, Br, OH, SCH₃ or a reactive esterified OH group,

is reacted with a compound of the formula III

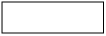

in which

R³, R⁴ and n are as defined above, or

b) a radical X in a compound of the formula I is converted into another radical X by, for example, hydrolysing an ester group to a COOH group or converting a COOH group into an amide or into a cyano group,

and/or in that a compound of the formula I is converted into one of its salts.

Above and below, the radicals R¹, R², R³, R⁴, R⁵, R⁶, R⁷, X, L and n are as defined under the formulae 1, 11 and III, unless expressly stated otherwise.

A is alkyl having 1-6 carbon atoms.

In the above formulae, alkyl is preferably unbranched and has 1, 2, 3, 4, 5 or 6 carbon atoms and is preferably methyl, ethyl or propyl, furthermore preferably isopropyl, butyl, isobutyl, sec-butyl or tert-butyl, but also n-pentyl, neopentyl, isopentyl or hexyl.

X is an R⁵ or R⁶ radical which is monosubstituted by R⁷—.

R⁵ is a linear or branched alkylene radical having 1-10 carbon atoms, preferably 1-8 carbon atoms, where the alkylene radical is preferably, for example, methylene, ethylene, propylene, isopropylene, butylene, isobutylene, sec-butylene, pentylene, 1-, 2- or 3-methylbutylene, 1,1-, 1,2- or 2,2-dimethylpropylene, 1-ethylpropylene, hexylene, 1-2-, 3- or 4-methylpentylene, 1,1-, 1,2-, 1,3-, 2,2-, 2,3- or 3,3-dimethylbutylene, 1- or 2-ethylbutylene, 1-ethyl-1-methylpropylene, 1-ethyl-2-methylpropylene, 1,1,2- or 1,2,2-trimethylpropylene, linear or branched heptylene, octylene, nonylene or decylene.

R⁵ is furthermore, for example, but-2-enylene or hex-3-enylene

R⁶ is cycloalkylalkylene having 6-12 carbon atoms, preferably, for example, cyclopentylmethylene, cyclohexylmethylene, cyclohexylethylene, cyclohexylpropylene or cyclohexylbutylene.

Of the radicals R¹ and R², one is preferably H, while the other is preferably propyl or butyl, but particularly preferably ethyl or methyl. Furthermore, R¹ and R² together are also preferably propylene, butylene or pentylene.

Hal is preferably F, Cl or Br, but also I.

The radicals R³ and R⁴ may be identical or different and are preferably located in the 3- or 4-position of the phenyl ring. They are, for example, in each case independently of one another, H, alkyl, F, Cl, Br or I or together are alkylene, such as, for example, propylene, butylene or pentylene, furthermore ethyleneoxy, methylenedioxy or ethylenedioxy. They are preferably also in each case alkoxy, such as, for example, methoxy, ethoxy or propoxy, furthermore also hydroxyl.

The radical R¹ is preferably, for example, COOH, COOCH₃, COOC₂H₅, CONH₂, CON(CH₃)₂, CONHCH₃ or CN.

For the entire invention, all radicals which occur more than once may be identical or different, i.e. are independent of one another.

Accordingly, the invention relates in particular to the compounds of the formula I in which at least one of the said radicals has one of the preferred meanings indicated above. Some preferred groups of compounds may be expressed by the following sub-formulae Ia to Id, which conform to the formula I and in which the radicals not designated in greater detail are as defined under the formula I, but in which

in Ia X is R⁵ or R⁶ which is substituted by COOH or COOA;

in Ib R¹ and R² are each, independently of one another, H, A or Hal, where at least one of the radicals R¹ or R² is always ≠H,

R³ and R⁴ together are alkylene having 3-5 carbon atoms, —O—CH₂—CH₂—, —O—CH₂—O— or —O—CH₂—CH₂—O,

X is R⁵ or R⁶ which is substituted by COOH or COOA;

in Ic R¹ and R² are each, independently of one another, H, A or Hal, where at least one of the radicals R¹ or R² is always ≠H,

R³ and R⁴ are each, independently of one another, H, A, OA or Hal,

R³ and R⁴ together are alkylene having 3-5 carbon atoms, —O—CH₂—CH₂—, —O—CH₂—O— or —O—CH₂—CH₂—O,

X is R⁵ or R⁶ which is substituted by COOH or COOA,

n is 1 or 2;

in Id R¹ and R² are each, independently of one another, H, A or Hal, where one of the radicals R¹ and R² is always ≠H,

R¹ and R² together are alternatively alkylene having 3-5 carbon atoms,

R³ and R⁴ are each, independently of one another, H, A, OA, OH or Hal,

R³ and R⁴ together are alternatively —O—CH₂—O—,

X is R⁵ which is monosubstituted by R⁷,

R⁵ is linear or branched alkylene having 1-10 carbon atoms or —C₆H₄—CH₂—,

R⁷ is COOH or COOA,

A is alkyl having from 1 to 6 carbon atoms,

Hal is F, Cl, Br or I ,

m is 1, and

n is 1 or 2.

The compounds of the formula I and also the starting materials for their preparation are, in addition, prepared by methods known per se, as described in the literature (for example in the standard works, such as Houben-Weyl, Methoden der organischen Chemie [Methods of Organic Chemistry], Georg-Thieme-Verlag, Stuttgart), to be precise under reaction conditions which are known and suitable for the said reactions. Use can also be made here of variants which are known per se, but are not mentioned here in greater detail.

In the compounds of the formula II or III , R¹, R², R³, R⁴, X and n have the meanings indicated, in particular the preferred meanings indicated.

If L is a reactive esterified OH group, this is preferably alkylsulfonyloxy having 1-6 carbon atoms (preferably methylsulfonyloxy) or arylsulfonyloxy having 6-10 carbon atoms (preferably phenyl- or p-tolylsulfonyloxy, further more also 2-naphthalenesulfonyloxy).

The compounds of the formula I can preferably be obtained by reacting compounds of the formula II with compounds of the formula III.

If desired, the starting materials can also be formed in situ by not isolating them from the reaction mixture, but instead immediately converting them further into the compounds of the formula I.

On the other hand, it is possible to carry out the reaction stepwise.

The starting compounds of the formulae II and III are generally known. If they are not known, they can be prepared by methods known per se. Compounds of the formula II can be obtained, for example, from compounds which are built up from thiophene derivatives and CN-substituted alkylenecarboxylic acid esters (Eur. J. Med. Chem. 23, 453 (1988)), by reaction with POCl₃.

In detail, the reaction of the compounds of the formula II with the compounds of the formula III is carried out in the presence or absence of an inert solvent at temperatures between about −20 and about 150°, preferably between 20 and 100°.

The addition of an acid-binding agent, for example an alkali or alkaline earth metal hydroxide, carbonate or bicarbonate or another salt of a weak acid of the alkali or alkaline earth metals, preferably of potassium, sodium or calcium, or the addition of an organic base, such as triethylamine, dimethylamine, pyridine or quinoline or of an excess of the amine component, may be favourable.

Examples of suitable inert solvents are hydrocarbons, such as hexane, petroleum ether, benzene, toluene or xylene; chlorinated hydrocarbons, such as trichloroethylene, 1,2-dichloroethane, tetrachloromethane, chloroform or dichloromethane; alcohols, such as methanol, ethanol, isopropanol, n-propanol, n-butanol or tert-butanol; ethers, such as diethyl ether, diisopropyl ether, tetrahydrofuran (THF) or dioxane; glycol ethers, such as ethylene glycol monomethyl or monoethyl ether or ethylene glycol dimethyl ether (diglyme); ketones, such as acetone or butanone; amides, such as acetamide, dimethylacetamide, N-methylpyrrolidone or dimethylformamide (DMF); nitriles, such as acetonitrile; sulfoxides, such as dimethyl sulfoxide (DMSO); nitro compounds, such as nitromethane or nitrobenzene; esters, such as ethyl acetate, or mixtures of the said solvents.

It is furthermore possible to convert a radical X in a compound of the formula I into another radical X, for example by hydrolysing an ester or a cyano group to give a COOH group.

Ester groups can be saponified, for example, using NaOH or KOH in water, water/THF or water/dioxane at temperatures between 0 and 100°. Carboxylic acids can be converted into the corresponding carboxylic acid chlorides, for example using thionyl chloride, and these can be converted into carboxamides. Elimination of water therefrom in a known manner gives carbonitriles.

An acid of the formula I can be converted into the associated acid-addition salt using a base, for example by reaction of equivalent amounts of the acid and the base in an inert solvent, such as ethanol, followed by evaporation. Suitable bases for this reaction are, in particular, those which give physiologically acceptable salts.

Thus, the acid of the formula I can be converted into the corresponding metal salt, in particular alkali metal or alkaline earth metal salt, or into the corresponding ammonium salt using a base (for example sodium hydroxide, potassium hydroxide, sodium carbonate or potassium carbonate).

Also suitable for this reaction are, in particular, organic bases which give physiologically acceptable salts, such as, for example, ethanolamine.

On the other hand, a base of the formula I can be converted into the associated acid-addition salt using an acid, for example by reaction of equivalent amounts of the base and the acid in an inert solvent, such as ethanol, followed by evaporation. Suitable acids for this reaction are, in particular, those which give physiologically acceptable acids. Thus, it is possible to use inorganic acids, for example sulfuric acid, nitric acid, hydrohalic acids, such as hydrochloric acid or hydrobromic acid, phosphoric acids, such as orthophosphoric acid, or sulfamic acid, furthermore organic acids, in particular aliphatic, alicyclic, araliphatic, aromatic or heterocyclic monobasic or polybasic carboxylic, sulfonic or sulfuric acids, for example formic acid, acetic acid, propionic acid, pivalic acid, diethylacetic acid, malonic acid, succinic acid, pimelic acid, fumaric acid, maleic acid, lactic acid, tartaric acid, malic acid, citric acid, gluconic acid, ascorbic acid, nicotinic acid, isonicotinic acid, methane- or ethanesulfonic acid, ethanedisulfonic acid, 2-hydroxyethanesulfonic acid, benzenesulfonic acid, p-toluenesulfonic acid, naphthalenemono- and -disulfonic acids, or laurylsulfuric acid. Salts with physiologically unacceptable acids, for example picrates, can be used for the isolation and/or purification of the compounds of the formula I.

The invention furthermore relates to the use of the compounds of the formula I and/or their physiologically acceptable salts for the production of pharmaceutical preparations, in particular by non-chemical methods. They can be converted into a suitable dosage form here together with at least one solid, liquid and/or semi-liquid excipient or assistant and optionally in combination with one or more further active ingredients.

The invention also relates to medicaments of the formula I and their physiologically acceptable salts as phosphodiesterase V inhibitors.

The invention furthermore relates to pharmaceutical preparations comprising at least one compound of the formula I and/or one of its physiologically acceptable salts.

These preparations can be used as medicaments in human or veterinary medicine. Suitable excipients are organic or inorganic substances which are suitable for enteral (for example oral), parenteral or topical administration and do no react with the novel compounds, for example water, vegetable oils, benzyl alcohols; alkylene glycols, polyethylene glycols, glycerol triacetate, gelatine, carbohydrates, such as lactose or starch, magnesium stearates, talc or vaseline. Suitable for oral administration are, in particular, tablets, pills, coated tablets, capsules, powders, granules, syrups, juices or drops, suitable for rectal administration are suppositories, suitable for parenteral administration are solutions, preferably oil-based or aqueous solutions, furthermore suspensions, emulsions or implants, and suitable for topical application are ointments, creams or powders. The novel compounds may also be lyophilised and the resultant lyophilisates used, for example, for the preparation of injection preparations. The preparations indicated may be sterilised and/or comprise assistants, such as lubricants, preservatives, stabilisers and/or wetting agents, emulsifiers, salts for modifying the osmotic pressure, buffer substances, colorants and flavours and/or a plurality of further active ingredients, for example one or more vitamins.

The compounds of the formula I and their physiologically acceptable salts can be employed for combating illnesses in which an increase in the cGMP (cycloguanosine monophosphate) level results in inflammation inhibition or prevention and muscle relaxation. The compounds according to the invention are used in particular in the treatment of illnesses of the cardiovascular system and for the treatment and/or therapy of impotence.

In general, the substances are preferably administered in doses of between about 1 and 500 mg, in particular between 5 and 100 mg per dosage unit. The daily dose is preferably between about 0.02 and 10 mg/kg of body weight. However, the specific dose for each patient depends on a wide variety of factors, for example on the efficacy of the specific compound employed, on the age, body weight, general state of health, sex, on the diet, on the time and method of administration, on the excretion rate, medicament combination and severity of the particular illness to which the therapy applies. Oral administration is preferred.

Above and below, all temperatures are given in ° C. In the examples below, “conventional work-up” means that water is added if necessary, a pH of from 2 to 10, depending on the constitution of the end product, is set if necessary, the mixture is extracted with ethyl acetate or dichloromethane, the phases are separated, the organic phase is dried over sodium sulfate and evaporated, and the product is purified by chromatography on silica gel and/or by crystallisation.

Mass spectrometry (MS): El (electron impact ionisation) M⁺

FAB (fast atom bombardment) (M+H)⁺

### EXAMPLE 1

1.9 g of methyl 3-(4-chloro-5,6,7,8-tetrahydro-[1]-benzothieno-[2,3-d]-pyrimidin-2-yl)propionate [obtainable by cyclisation of methyl 2-amino-4,5,6,7-tetrahydrobenzothiophene-3-carboxylate with methyl 3-cyanopropionate, followed by chlorination using phosphorus oxychloride/dimethylamine] and 2.3 g of 3-chloro-4-methoxybenzylamine (“A”) in 20 ml of N-methylpyrrolidone are stirred at 1100 for 5 hours. The solvent is removed, and the mixture is subjected to conventional work-up, giving 2.6 g of methyl 3-[4-(3-chloro-4-methoxybenzylamino)-5,6,7,8-tetrahydro-[1]-benzothieno-[2,3-d]-pyrimidin-2-yl]propionate as a colourless oil.

Analogous reaction of “A”

with methyl 3-(4-chloro-5,6-cyclopenteno-[1]-benzothieno-[2, 3-d]-pyrimidin-2-yl)propionate gives

methyl 3-[4-(3-chloro-4-methoxybenzylamino)-5,6-cyclopenteno-[1]-benzothieno-[2,3-d]-pyrimidin-2-yl]propionate;

with methyl 3-(4-chloro-5,6-cyclohepteno-[1]-benzothieno-[2,3-d]-pyrimidin-2-yl)propionate gives

methyl 3-[4-(3-chloro-4-methoxybenzylamino)-5,6-cyclohepteno-[1]-benzothieno-[2,3-d]-pyrimidin-2-yl]propionate;

with methyl 3-(4-chloro-6-methylthieno-[2,3-d]-pyrimidin-2-yl)propionate gives

methyl 3-[4-(3-chloro-4-methoxybenzylamino)-6-methylthieno-[2,3-d]-pyrimidin-2-yl]propionate;

with methyl 3-(4-chloro-5,6-dimethylthieno-[2,3-d]-pyrimidin-2-yl)propionate gives

methyl 3-[4-(3-chloro-4-methoxybenzylamino)-5,6-dimethylthieno-[2,3-d]-pyrimidin-2-yl]propionate;

with methyl 3-(4-chloro-6-ethylthieno-[2,3-d]-pyrimidin-2-yl)propionate gives

methyl 3-[4-(3-chloro-4-methoxybenzylamino)-6-ethylthieno-[2,3-d]-pyrimidin-2-yl]propionate;

with methyl 3-(4,6-Dichlorothieno-[2,3-d]-pyrimidin-2-yl)propionate gives

methyl 3-[4-(3-chloro-4-methoxybenzylamino)-6-chlorothieno-[2,3-d]-pyrimidin-2-yl]propionate;

with methyl 2-(4-chloro-5,6,7,8-tetrahydro-[1]-benzothieno-[2,3-d]-pyrimidin-2-yl)acetate gives

methyl 2-[4-(3-chloro-4-methoxybenzylamino)-5,6,7,8-tetrahydro-[1]-benzothieno-[2, 3-d]-pyrimidin-2-yl]acetate.

Analogous reaction of 3,4-methylenedioxybenzylamine

with methyl 3-(4-chloro-5,6,7,8-tetrahydro-[1]-benzothieno-[2,3-d]-pyrimidin-2-yl)propionate gives

methyl 3-[4-(3,4-methylenedioxybenzylamino)-5,6,7,8-tetrahydro-[1]-benzothieno-[2,3-d]-pyrimidin-2-yl]propionate;

with methyl 3-(4-chloro-5,6-cyclopenteno-[1]-benzothieno-[2,3-d]-pyrimidin-2-yl)propionate gives

3-[4-(3,4-methylenedioxybenzylamino)-5,6-cyclopenteno-[1]-benzothieno-[2, 3-d]-pyrimidin-2-yl]propionate;

with methyl 3-(4-chloro-5,6-cyclohepteno-[1]-benzothieno-[2,3-d]-pyrimidin-2-yl)propionate gives

methyl 3-[4-(3,4-methylenedioxybenzylamino)-5,6-cyclohepteno-[1]-benzothieno-[2,3-d]-pyrimidin-2-yl]propionate;

with methyl 3-(4-chloro-6-methylthieno-[2,3-d]-pyrimidin-2-yl)propionate gives

methyl 3-[4-(3,4-methylenedioxybenzylamino)-6-methylthieno-[2,3-d]-pyrimidin-2-yl]propionate;

with methyl 3-(4-chloro-5,6-dimethylthieno-[2,3-d]-pyrimidin-2-yl)propionate gives

methyl 3-[4-(3,4-methylenedioxybenzylamino)-5,6-dimethylthieno-[2,3-d]-pyrimidin-2-yl]propionate;

with methyl 3-(4-chloro-6-ethylthieno-[2,3-d]-pyrimidin-2-yl)propionate gives

methyl 3-[4-(3,4-methylenedioxybenzylamino)-6-ethylthieno-[2,3-d]-pyrimidin-2-yl]propionate;

with methyl 3-(4,6-dichlorothieno-[2,3-d]-pyrimidin-2-yl)propionate gives

methyl 3-[4-(3,4-methylenedioxybenzylamino)-6-chlorothieno-[2,3-d]-pyrimidin-2-yl]propionate.

Analogous reaction of “A”

with methyl 4-(4-chloro-5,6,7,8-tetrahydro-[1]-benzothieno-[2,3-d]-pyrimidin-2-yl)butyrate gives

methyl 4-[4-(3-chloro-4-methoxybenzylamino)-5,6,7,8-tetrahydro-[1]-benzothieno-[2,3-d]-pyrimidin-2-yl]butyrate;

with methyl 4-(4-chloro-5,6-cyclopenteno-[1]-benzothieno-[2,3-d]-pyrimidin-2-yl)butyrate gives.

4-[4-(3-chloro-4-methoxybenzylamino)-5,6-cyclopenteno-[1]-benzothieno-[2,3-d]-pyrimidin-2-yl]butyrate;

with methyl 4-(4-chloro-5,6-cyclohepteno-[1]-benzothieno-[2,3-d]-pyrimidin-2-yl)butyrate gives

methyl 4-[4-(3-chloro-4-methoxybenzylamino)-5,6-cyclohepteno-[1]-benzothieno-[2, 3-d]-pyrimidin-2-yl]butyrate;

with methyl 4-(4-chloro-6-methylthieno-[2,3-d]-pyrimidin-2-yl)butyrate gives

methyl 4-[4-(3-chloro-4-methoxybenzylamino)-6-methylthieno-[2,3-d]-pyrimidin-2-yl]butyrate;

with methyl 4-(4-chloro-5,6-dimethylthieno-[2,3-d]-pyrimidin-2-yl)butyrate gives

methyl 4-[4-(3-chloro-4-methoxybenzylamino)-5,6-dimethylthieno-[2,3-d]-pyrimidin-2-yl]butyrate;

with methyl 4-(4-chloro-4-ethylthieno-[2,3-d]-pyrimidin-2-yl)butyrate gives

methyl 4-[4-(3-chloro-4-methoxybenzylamino)-6-ethylthieno-[2,3-d]-pyrimidin-2-yl]butyrate;

with methyl 4-(4,6-chloro-6-chlorothieno-[2,3-d]-pyrimidin-2-yl)butyrate gives

methyl 4-[4-(3-chloro-4-methoxybenzylamino)6-chlorothieno-[2,3-d]-pyrimidin-2-yl]butyrate.

Analogous reaction of 3,4-methylenedioxybenzylamine

with methyl 4-(4-chloro-5,6,7,8-tetrahydro-[1]-benzothieno-[2,3-d]-pyrimidin-2-yl)butyrate gives

methyl 4-[4-(3,4-methylenedioxybenzylamino)-5,6,7,8-tetrahydro-[1]-benzothieno-[2,3-d]-pyrimidin-2-yl]butyrate;

with methyl 4-(4-chloro-5,6-cyclopenteno-[1]-benzothieno-[2,3-d]-pyrimidin-2-yl)butyrate gives

4-[4-(3,4-methylenedioxybenzylamino)-5,6-cyclopenteno-[1]-benzothieno-[2,3-d]-pyrimidin-2-yl]butyrate;

with methyl 4-(4-chloro-5,6-cyclohepteno-[1]-benzothieno-[2,3-d]-pyrimidin-2-yl)butyrate gives

methyl 4-[4-(3,4-methylenedioxybenzylamino)-5,6-cyclohepteno-[1]-benzothieno-[2,3-d]-pyrimidin-2-yl]butyrate;

with methyl 4-(4-chloro-6-methylthieno-[2,3-d]-pyrimidin-2-yl)butyrate gives

methyl 4-[4-(3,4-methylenedioxybenzylamino)-6-methylthieno-[2,3-d]-pyrimidin-2-yl]butyrate;

with methyl 4-(4-chloro-5,6-dimethylthieno-[2,3-d]-pyrimidin-2-yl)butyrate gives

methyl 4-[4-(3,4-methylenedioxybenzylamino)-5,6-dimethylthieno-[2,3-d]-pyrimidin-2-yl]butyrate;

with methyl 4-(4-chloro-6-ethylthieno-[2,3-d]-pyrimidin-2-yl)butyrate gives

methyl 4-[4-(3,4-methylenedioxybenzylamino)-6-ethylthieno-[2,3-d]-pyrimidin-2-yl]butyrate;

with methyl 4-(4,6-dichlorothieno-[2,3-d]-pyrimidin-2-yl)butyrate gives

methyl 4-[4-(3,4-methylenedioxybenzylamino)-6-chlorothieno-[2, 3-d]-pyrimidin-2-yl]butyrate.

Analogous reaction of “A”

with methyl 5-(4-chloro-5,6,7,8-tetrahydro-[1]-benzothieno-[2,3-d]-pyrimidin-2-yl)valerate gives

methyl 5-[4-(3-chloro-4-methoxybenzylamino)-5,6,7,8-tetrahydro-[1]-benzothieno-[2,3-d]-pyrimidin-2-yl]valerate;

with methyl 5-(4-chloro-5,6-cyclopenteno-[1]-benzothieno-[2,3-d]-pyrimidin-2-yl)valeric acid gives

5-[4-(3-chloro-4-methoxybenzylamino)-5,6-cyclopenteno-[1]-benzothieno-[2, 3-d]-pyrimidin-2-yl]valerate;

with methyl 5-(4-chloro-5,6-cyclohepteno-[1]-benzothieno-[2,3-d]-pyrimidin-2-yl) valeric acid gives

methyl 5-[4-(3-chloro-4-methoxybenzylamino)-5,6-cyclohepteno-[1]-benzothieno-[2,3-d]-pyrimidin-2-yl]valerate;

with methyl 5-(4-chloro-6-methylthieno-[2,3-d]-pyrimidin-2-yl)valerate gives

methyl 5-[4-(3-chloro-4-methoxybenzylamino)-6-methylthieno-[2,3-d]-pyrimidin-2-yl]valerate;

with methyl 5-(4-chloro-5,6-dimethylthieno-[2,3-d]-pyrimidin-2-yl)valerate gives

methyl 5-[4-(3-chloro-4-methoxybenzylamino)-5,6-dimethylthieno-[2, 3-d]-pyrimidin-2-yl]valerate;

with methyl 5-(4-chloro-6-ethylthieno-[2,3-d]-pyrimidin-2-yl)valerate gives

methyl 5-[4-(3-chloro-4-methoxybenzylamino)-6-ethylthieno-[2,3-d]-pyrimidin-2-yl]valerate;

with methyl 5-(4,6-dichlorothieno-[2,3-d]-pyrimidin-2-yl)valerate gives

methyl 5-[4-(3-chloro-4-methoxybenzylamino)-6-chlorothieno-[2,3-d]-pyrimidin-2-yl]valerate.

Analogous reaction of 3,4-methylenedioxybenzylamine

with methyl 5-(4-chloro-5,6,7,8-tetrahydro-[1]-benzothieno-[2,3-d]-pyrimidin-2-yl)valerate gives

methyl 5-[4-(3,4-methylenedioxybenzylamino)-5,6,7,8-tetrahydro-[1]-benzothieno-[2, 3-d]-pyrimidin-2-yl]valerate;

with methyl 5-(4-chloro-5,6-cyclopenteno-[1]-benzothieno-[2,3-d]-pyrimidin-2-yl)valerate gives

5-[4-(3,4-methylenedioxybenzylamino)-5,6-cyclopenteno-[1]-benzothieno-[2, 3-d]-pyrimidin-2-yl]valerate;

with methyl 5-(4-chloro-5,6-cyclohepteno-[1]-benzothieno-[2,3-d]-pyrimidin-2-yl)valerate gives

methyl 5-[4-(3,4-methylenedioxybenzylamino)-5,6-cyclohepteno-[l]-benzothieno-[2,3-d]-pyrimidin-2-yl]valerate;

with methyl 5-(4-chloro-6-methylthieno-[2,3-d]-pyrimidin-2-yl)valerate gives

methyl 5-[4-(3,4-methylenedioxybenzylamino)-6-methylthieno-[2,3-d]-pyrimidin-2-yl]valerate;

with methyl 5-(4-chloro-5,6-dimethylthieno-[2,3-d]-pyrimidin-2-yl)valerate gives

methyl 5-[4-(3,4-methylenedioxybenzylamino)-5,6-dimethylthieno-[2,3-d]-pyrimidin-2-yl]valerate;

with methyl 5-(4-chloro-6-ethylthieno-[2,3-d]-pyrimidin-2-yl)valerate gives

methyl 5-[4-(3, 4-methylenedioxybenzylamino)-6-ethylthieno-[2, 3-d]-pyrimidin-2-yl]valerate;

with methyl 5-(4,6-dichlorothieno-[2,3-d]-pyrimidin-2-yl)valerate gives

methyl 5-[4-(3,4-methylenedioxybenzylamino)-6-chlorothieno-[2,3-d]-pyrimidin-2-yl]valerate.

Analogous reaction of “A”

with methyl 7-(4-chloro-5,6,7,8-tetrahydro-[1]-benzothieno-[2,3-d]-pyrimidin-2-yl)heptanoate

methyl 7-[4-(3-chloro-4-methoxybenzylamino)-5,6,7,8-tetrahydro-[1]-benzothieno-[2,3-d]-pyrimidin-2-yl]heptanoate;

with methyl 7-(4-chloro-5,6-cyclopenteno-[1]-benzothieno-[2,3-d]-pyrimidin-2-yl)-heptanoate gives

methyl 7-[4-(3-chloro-4-methoxybenzylamino)-5,6-cyclopenteno-[1]-benzothieno-[2,3-d]-pyrimidin-2-yl]heptanoate;

with methyl 7-(4-chloro-5,6-cyclohepteno-[l]-benzothieno-[2,3-d]-pyrimidin-2-yl)-heptanoate gives

methyl 7-[4-(3-chloro-4-methoxybenzylamino)-5,6-cyclohepteno-[1]-benzothieno-[2,3-d]-pyrimidin-2-yl]heptanoate; with methyl 7-(4-chloro-6-methylthieno-[2,3-d]-pyrimidin-2-yl)heptanoate gives

methyl 7-[4-(3-chloro-4-methoxybenzylamino)-6-methylthieno-[2,3-d]-pyrimidin-2-yl]heptanoate;

with methyl 7-(4-chloro-5,6-dimethylthieno-[2,3-d]-pyrimidin-2-yl)heptanoate gives

methyl 7-[4-(3-chloro-4-methoxybenzylamino)-5,6-dimethylthieno-[2,3-d]-pyrimidin-2-yl]heptanoate;

with methyl 7-(4-chloro-6-ethylthieno-[2,3-d]-pyrimidin-2-yl)heptanoate gives

methyl 7-[4-(3-chloro-4-methoxybenzylamino)-6-ethylthieno-[2,3-d]-pyrimidin-2-yl]heptanoate;

with methyl 7-(4-chloro-6-chlorothieno-[2,3-d]-pyrimidin-2-yl)heptanoate gives

methyl 7-[4-(3-chloro-4-methoxybenzylamino)-6-chlorothieno-[2,3-d]-pyrimidin-2-yl)heptanoate.

Analogous reaction of 3,4-methylenedioxybenzylamine

with methyl 7-(4-chloro-5,6,7,8-tetrahydro-[1]-benzothieno-[2, 3-d]-pyrimidin-2-yl)heptanoate gives

methyl 7-[4-(3,4-methylenedioxybenzylamino)-5,6,7,8-tetrahydro-[1]-benzothieno-[2,3-d]-pyrimidin-2-yl]heptanoate;

with methyl 7-(4-chloro-5,6-cyclopenteno-[1]-benzothieno-[2,3-d]-pyrimidin-2-yl)heptanoate gives

methyl 7-[4-(3,4-methylenedioxybenzylamino)-5,6-cyclopenteno-[1]-benzothieno-[2,3-d]-pyrimidin-2-yl]heptanoate;

with methyl 7-(4-chloro-5,6-cyclohepteno-[1]-benzothieno-[2,3-d]-pyrimidin-2-yl)heptanoate gives

methyl 7-[4-(3,4-methylenedioxybenzylamino)-5,6-cyclohepteno-[1]-benzothieno-[2,3-d]-pyrimidin-2-yl]heptanoate;

with methyl 7-(4-chloro-6-methylthieno-[2,3-d]-pyrimidin-2-yl)heptanoate gives

methyl 7-[4-(3,4-methylenedioxybenzylamino)-6-methylthieno-[2,3-d]-pyrimidin-2-yl]valerate;

with methyl 7-(4-chloro-5,6-dimethylthieno-[2,3-d]-pyrimidin-2-yl)heptanoate gives

methyl 7-[4-(3,4-methylenedioxybenzylamino)-5,6-dimethylthieno-[2,3-d]-pyrimidin-2-yl]heptanoate;

with methyl 7-(4-chloro-6-ethylthieno-[2,3-d]-pyrimidin-2-yl)heptanoate gives

methyl 7-[4-(3,4-methylenedioxybenzylamino)-6-ethylthieno-[2,3-d]pyrimidin-2-yl]heptanoate;

with methyl 7-(4,6-dichlorothieno-[2,3-d]-pyrimidin-2-yl)heptanoate gives

methyl 7-[4-(3,4-methylenedioxybenzylamino)-6-chlorothieno-[2,3-d]-pyrimidin-2-yl]heptanoate.

Analogous reaction of “A”

with methyl 2-[4-(4-chloro-5,6,7,8-tetrahydro-[1]-benzothieno-[2, 3-d]-pyrimidin-2-yl)-cyclohexyl-1-yl]acetate gives

methyl 2{-4-[4-(3-chloro-4-methoxybenzylamino)-5,6,7,8-tetrahydro-[1]-benzothieno-[2,3-d]-pyrimidin-2-yl]-cyclohexyl-1-yl}acetate;

with methyl 2-(4-(4-chloro-4-ethylthieno-[2,3-d]-pyrimidin-2-yl)-cyclohexyl-1-yl]acetate gives

methyl 2{-4-[4-(3-chloro-4-methoxybenzylamino)-6-ethylthieno-[2, 3-d]-pyrimidin-2-yl]-cyclohexyl-1-yl}acetate;

Analogous reaction of 3,4-methylenedioxybenzylamine

with methyl 2-[4-(4-chloro-5,6,7,8-tetrahydro-[1]-benzothieno-[2,3-d]-pyrimidin-2-yl)-cyclohexyl-1-yl]acetate gives

methyl 2{-4-[4-(3,4-methylenedioxybenzylamino)-5,6,7,8-tetrahydro-[1]-benzothieno-[2, 3-d]-pyrimidin-2-yl]-cyclohexyl-1-yl}acetate.

Analogous reaction of benzylamine

with methyl 3-(4-chloro-5,6,7,8-tetrahydro-[1]-benzothieno-[2,3-d]-pyrimidin-2-yl)propionate gives

methyl 3-(4-benzylamino-5,6,7,8-tetrahydro-[1]-benzothieno-[2,3-d]-pyrimidin-2-yl)propionate;

with methyl 4-(4-chloro-5,6,7,8-tetrahydro-[1]-benzothieno-[2,3-d]-pyrimidin-2-yl)butyrate gives

methyl 4-(4-benzylamino-5,6,7,8-tetrahydro-[1]-benzothieno-[2,3-d]-pyrimidin-2-yl)butyrate;

with methyl 5-(4-chloro-5,6,7,8-tetrahydro-[1]-benzothieno-[2,3-d]-pyrimidin-2-yl)valerate gives

methyl 5-(4-benzylamino-5,6,7,8-tetrahydro-[1]-benzothieno-[2,3-d]-pyrimidin-2-yl)valerate;

with methyl 4-(4-chloro-6-methylthieno-[2,3-d]-pyrimidin-2-yl)butyrate gives

methyl 4-[4-benzylamino-6-methylthieno-[2,3-d]-pyrimidin-2-yl]-butyrate;

with methyl 5-(4-chloro-6-ethylthieno-[2,3-d]-pyrimidin-2-yl)valerate gives

methyl 5-[4-benzylamino-6-ethylthieno-[2,3-d]-pyrimidin-2-yl]valerate.

### EXAMPLE 2

2.2 g of methyl 3-[4-(3-chloro-4-methoxybenzylamino)-5,6,7,8-tetrahydro-[1]-benzothieno-[2,3-d]-pyrimidin-2-yl]propionate are dissolved in 20 ml of ethylene glycol monomethyl ether, 10 ml of 32% NaOH are added, and the mixture is stirred at 110° for 5 hours. 20% HCl is added, and the mixture is extracted with dichloromethane. Addition of petroleum ether gives 2.0 g of 3-[4-(3-chloro-4-methoxybenzylamino)-5,6,7,8-tetrahydro-[1]-benzothieno-[2,3-d]-pyrimidin-2-yl]propionic acid, m.p. 229°.

The deposited crystals are dissolved in 30 ml of isopropanol, and 0.5 g of ethanolamine is added. Crystallisation gives 1.35 g of 3-[4-(3-chloro-4-methoxybenzylamino)-5,6,7,8-tetrahydro-[1]-benzothieno-[2,3-d]-pyrimidin-2-yl]propionic acid, ethanolamine salt, m.p. 1350.

Analogous reaction of the esters listed under Example 1 gives the following carboxylic acids:

3-[4-(3-chloro-4-methoxybenzylamino)-5,6-cyclopenteno-[1]-benzothieno-[2,3-d]-pyrimidin-2-yl]propionic acid;

3-[4-(3-chloro-4-methoxybenzylamino)-5,6-cyclohepteno-[1]-benzothieno-[2,3-d]-pyrimidin-2-yl]propionic acid;

3-[4-(3-chloro-4-methoxybenzylamino)-6-methylthieno-[2,3-d]-pyrimidin-2-yl]propionic acid;

3-[4-(3-chloro-4-methoxybenzylamino)-5,6-methylthieno-[2,3-d]-pyrimidin-2-yl]propionic acid;

3-[4-(3-chloro-4-methoxybenzylamino)-6-ethylthieno-[2,3-d]-pyrimidin-2-yl]propionic acid;

3-[4-(3-chloro-4-methoxybenzylamino)-6-chlorothieno-[2, 3-d]-pyrimidin-2-yl]propionic acid;

2-[4-(3-chloro-4-methoxybenzylamino)-5,6,7,8-tetrahydro-[1]-benzothieno-[2,3-d]-pyrimidin-2-yl]acetic acid, ethanolamine salt, m.p. 126°;

3-[4-(3,4-methylenedioxybenzylamino)-5,6,7,8-tetrahydro-[1]-benzothieno-[2,3-d]-pyrimidin-2-yl]propionic acid;

3-[4-(3,4-methylenedioxybenzylamino)-5,6-cyclopenteno-[1]-benzothieno-[2,3-d]-pyrimidin-2-yl]propionic acid;

3-[4-(3,4-methylenedioxybenzylamino)-5,6-cyclohepteno-[1]-benzothieno-[2,3-d]-pyrimidin-2-yl]propionic acid;

3-[4-(3,4-methylenedioxybenzylamino)-6-methylthieno-[2, 3-d]-pyrimidin-2-yl]propionic acid;

3-[4-(3,4-methylenedioxybenzylamino)-5,6-dimethylthieno-[2,3-d]-pyrimidin-2-yl]propionic acid;

3-[4-(3,4-methylenedioxybenzylamino)6-ethylthieno-[2,3-d]-pyrimidin-2-yl]propionic acid;

3-[4-(3,4-methylenedioxybenzylamino)-6-chlorothieno-[2,3-d]-pyrimidin-2-yl]propionic acid;

4-[4-(3-chloro-4-methoxybenzylamino)-5,6,7,8-tetrahydro-[1]-benzothieno-[2,3-d]-pyrimidin-2-yl]butyric acid;

4-[4-(3-chloro-4-methoxybenzylamino)-5,6-cyclopenteno-[1]-benzothieno-[2,3-d]-pyrimidin-2-yl]butyric acid;

4-[4-(3-chloro-4-methoxybenzylamino)-5,6-cyclohepteno-[1]-benzothieno-[2,3-d]-pyrimidin-2-yl]butyric acid;

4-[4-(3-chloro-4-methoxybenzylamino)-6-methylthieno-[2,3-d]-pyrimidin-2-yl]butyric acid, ethanolamine salt, m.p. 142°;

4-[4-(3-chloro-4-methoxybenzylamino)-5,6-methylthieno-[2,3-d]-pyrimidin-2-yl]butyric acid;

4-[4-(3-chloro-4-methoxybenzylamino)-6-ethylthieno-[2,3-d]-pyrimidin-2-yl]butyric acid, ethanolamine salt, m.p. 170°;

4-[4-(3-chloro-4-methoxybenzylamino)-6-chlorothieno-[2,3-d]-pyrimidin-2-yl]butyric acid;

4-[4-(3,4-methylenedioxybenzylamino)-5,6,7,8-tetrahydro-[1]-benzothieno-[2,3-d]-pyrimidin-2-yl]butyric acid, ethanolamine salt, m.p. 114°;

4-[4-(3,4-methylenedioxybenzylamino)-5,6-cyclopenteno-[1]-benzothieno-[2,3-d]-pyrimidin-2-yl]butyric acid;

4-[4-(3,4-methylenedioxybenzylamino)-5,6-cyclohepteno-[1]-benzothieno-[2,3-d]-pyrimidin-2-yl]butyric acid;

4-[4-(3,4-methylenedioxybenzylamino)-6-methylthieno-[2,3-d]-pyrimidin-2-yl]butyric acid, ethanolamine salt, m.p. 170°;

4-[4-(3,4-methylenedioxybenzylamino)-5,6-dimethylthieno-[2,3-d]-pyrimidin-2-yl]butyric acid;

4-[4-(3,4-methylenedioxybenzylamino)-6-ethylthieno-[2,3-d]-pyrimidin-2-yl]butyric acid;

4-[4-(3,4-methylenedioxybenzylamino)-6-chlorothieno-[2,3-d]-pyrimidin-2-yl]butyric acid;

5-[4-(3-chloro-4-methoxybenzylamino)-5,6,7,8-tetrahydro-[1]-benzothieno-[2,3-d]-pyrimidin-2-yl]valeric acid, m.p. 165°; ethanolamine salt, m.p. 112°;

5-[4-(3-chloro-4-methoxybenzylamino)-5,6-cyclopenteno-[1]-benzothieno-[2,3-d]-pyrimidin-2-yl]valeric acid;

5-[4-(3-chloro-4-methoxybenzylamino)-5,6-cyclohepteno-[1]-benzothieno-[2,3-d]-pyrimidin-2-yl]valeric acid;

5-[4-(3-chloro-4-methoxybenzylamino)-6-methylthieno-[2,3-d]-pyrimidin-2-yl]valeric acid, ethanolamine salt, m.p. 156°;

5-[4-(3-chloro-4-methoxybenzylamino)-5,6-dimethylthieno-[2,3-d]-pyrimidin-2-yl]valeric acid;

5-[4-(3-chloro-4-methoxybenzylamino)-6-ethylthieno-[2,3-d]-pyrimidin-2-yl]valeric acid, ethanolamine salt, m.p. 156°;

5-[4-(3-chloro-4-methoxybenzylamino)-6-chlorothieno-[2,3-d]-pyrimidin-2-yl]valeric acid;

5-[4-(3,4-methylenedioxybenzylamino)-5,6,7,8-tetrahydro-[1]-benzothieno-[2,3-d]-pyrimidin-2-yl]valeric acid;

5-[4-(3,4-methylenedioxybenzylamino)-5,6-cyclopenteno-[1]-benzothieno-[2,3-d]-pyrimidin-2-yl]valeric acid;

5-[4-(3,4-methylenedioxybenzylamino)-5,6-cyclohepteno-[1]-benzothieno-[2,3-d]-pyrimidin-2-yl]valeric acid;

5-[4-(3,4-methylenedioxybenzylamino)-6-methylthieno-[2,3-d]-pyrimidin-2-yl]valeric acid, ethanolamine salt, m.p. 167°;

5-[4-(3,4-methylenedioxybenzylamino)-5,6-dimethylthieno-[2,3-d]-pyrimidin-2-yl]valeric acid;

5-[4-(3,4-methylenedioxybenzylamino)-6-ethylthieno-[2,3-d]-35 pyrimidin-2-yl]valeric acid;

5-[4-(3,4-methylenedioxybenzylamino)-6-chlorothieno-[2,3-d]-pyrimidin-2-yl]valeric acid;

7-[4-(3-chloro-4-methoxybenzylamino)-5,6,7,8-tetrahydro-[1]-benzothieno-[2,3-d]-pyrimidin-2-yl]heptanoic acid, ethanolamine salt, m.p. 130°;

7-[4-(3-chloro-4-methoxybenzylamino)-5,6-cyclopenteno-[1]-benzothieno-[2,3-d]-pyrimidin-2-yl]heptanoic acid;

7-[4-(3-chloro-4-methoxybenzylamino)-5,6-cyclohepteno-[1]-benzothieno-[2,3-d]-pyrimidin-2-yl]heptanoic acid;

7-[4-(3-chloro-4-methoxybenzylamino)-6-methylthieno-[2,3-d]-pyrimidin-2-yl]heptanoic acid;

7-[4-(3-chloro-4-methoxybenzylamino)-5,6-dimethylthieno-[2,3-d]-pyrimidin-2-yl]heptanoic acid;

7-[4-(3-chloro-4-methoxybenzylamino)-6-ethylthieno-[2,3-d]-pyrimidin-2-yl]heptanoic acid;

7-[4-(3-chloro-4-methoxybenzylamino)-6-chlorothieno-[2,3-d]-pyrimidin-2-yl]heptanoic acid;

7-[4-(3,4-methylenedioxybenzylamino)-5,6,7,8-tetrahydro-[1]-benzothieno-[2,3-d]-pyrimidin-2-yl]heptanoic acid, ethanolamine salt, m.p. 137°;

7-[4-(3,4-methylenedioxybenzylamino)-5,6-cyclopenteno-[1]-benzothieno-[2,3-d]-pyrimidin-2-yl]heptanoic acid;

7-[4-(3,4-methylenedioxybenzylamino)-5,6-cyclohepteno-[1]-benzothieno-[2,3-d]-pyrimidin-2-yl]heptanoic acid;

7-[4-(3,4-methylenedioxybenzylamino)-6-methylthieno-[2,3-d]-pyrimidin-2-yl]valeric acid;

7-[4-(3,4-methylenedioxybenzylamino)-5,6-dimethylthieno-[2,3-d]-pyrimidin-2-yl]heptanoic acid;

7-[4-(3,4-methylenedioxybenzylamino)-6-ethylthieno-[2,3-d]-pyrimidin-2-yl]heptanoic acid;

7-[4-(3,4-methylenedioxybenzylamino)-6-chlorothieno-[2,3-d]-pyrimidin-2-yl]heptanoic acid;

2{4-[4-(3-chloro-4-methoxybenzylamino)-5,6,7,8-tetrahydro-[1]-benzothieno-[2,3-d]-pyrimidin-2-yl]cyclohexyl}acetic acid;

2{4-[4-(3-chloro-4-methoxybenzylamino)-6-ethylthieno-[2,3-d]-pyrimidin-2-yl]cyclohexyl}acetic acid;

2-{4-[4-(3,4-methylenedioxybenzylamino)-5,6,7,8-tetrahydro-[1]-benzothieno-[2,3-d]-pyrimidin-2-yl]cyclohexyl}acetic acid;

3-(4-benzylamino-5,6,7,8-tetrahydro-[1]-benzothieno-[2,3-d]-pyrimidin-2-yl)propionic acid, ethanolamine salt, m.p. 126°;

4-(4-benzylamino-5,6,7,8-tetrahydro-[1]-benzothieno-[2,3-d]-pyrimidin-2-yl)butyric acid, ethanolamine salt, m.p. 133°;

5-(4-benzylamino-5,6,7,8-tetrahydro-[1]-benzothieno-[2,3-d]-pyrimidin-2-yl)valeric acid, ethanolamine salt, m.p. 135°;

4-[4-benzylamino-6-methylthieno-[2,3-d]-pyrimidin-2-yl]butyric acid, ethanolamine salt, m.p. 165°;

5-[4-benzylamino-6-ethylthieno-[2,3-d]-pyrimidin-2-yl]valeric acid, ethanolamine salt, m.p. 162°.

### EXAMPLE 3

1 equivalent of 3-[4-(3-chloro-4-methoxybenzylamino)-5,6,7,8-tetrahydro-[1]-benzothieno-[2,3-d]-pyrimidin-2-yl]propionic acid and 1.2 equivalents of thionyl chloride are stirred in dichloromethane for 2 hours. The solvent is removed, giving 3-[4-(3-chloro-4-methoxybenzylamino)-5,6,7,8-tetrahydro-[1]-benzothieno-[2,3-d]-pyrimidin-2-yl]propionyl chloride.

The product is transferred into aqueous ammonia, and the mixture is stirred for one hour and subjected to conventional work-up, giving 3-[4-(3-chloro-4-methoxybenzylamino)-5,6,7,8-tetrahydro-[1]-benzothieno-[2,3-d]-pyrimidin-2-yl]propionamide.

### EXAMPLE 4

1 equivalent of DMF and 1 equivalent of oxalyl chloride are dissolved in acetonitrile at 00.1 equivalent of 3-[4-(3-chloro-4-methoxybenzylamino)-5,6,7,8-tetrahydro-[1]-benzothieno-[2,3-d]-pyrimidin-2-yl]-propionamide is then added. The mixture is stirred for a further hour. Conventional work-up gives 3-[4-(3-chloro-4-methoxybenzylamino)-5,6,7,8-tetrahydro-[1]-benzothieno-[2,3-d]-pyrimidin-2-yl]propionitrile.

### EXAMPLE 5

The following compounds are obtained analogously to Examples 1 and 2

6-[4-(3-chloro-4-methoxybenzylamino)-5,6,7,8-tetrahydro-[1]-benzothieno-[

2,3-d]-pyrimidin-2-yl]hexanoic acid, m.p. 165°;

2-[4-(3-chloro-4-methoxybenzylamino)-5,6,7,8-tetrahydro-[1]-benzothieno-[2,3-d]-pyrimidin-2-yl]propionic acid, ethanolamine salt, m.p. 150°;

4-[4-(3-chloro-4-methoxybenzylamino)-5,6,7,8-tetrahydro-[1]-benzothieno-[2,3-d]-pyrimidin-2-yl]-2,2-dimethylbutyric acid, ethanolamine salt, m.p. 130°;

4-(4-(3,4-methylenedioxybenzylamino)-5,6,7,8-tetrahydro-[1]-benzothieno-[[2,3-d]-pyrimidin-2-yl]-2,2-dimethylbutyric acid, ethanolamine salt, m.p. 126°;

5-[4-(3-chloro-4-hydroxybenzylamino)-5,6,7,8-tetrahydro-[1]-benzothieno-[2,3-d]-pyrimidin-2-yl]valeric acid, m.p. 179°;

5-[4-(3,4-dichlorobenzylamino)-5,6,7,8-tetrahydro-[1-benzothieno-[2,3-d]-pyrimidin-2-yl]valeric acid, ethanolamine salt, m.p. 136°;

5-[4-(3-chloro-4-isopropyloxybenzylamino)-5,6,7,8-tetrahydro-1]-benzothieno-[2,3-d]-pyrimidin-2-yl]valeric acid, ethanolamine salt, m.p. 1180;

2-[4-(4-(3-chloro-4-methoxybenzylamino)-5,6,7,8-tetrahydro-[1]-benzothieno-[2,3-d]-pyrimidin-2-yl)-phenyl]acetic acid, ethanolamine salt, m.p. 119°;

2-[4-(4-(3,4-methylenedioxybenzylamino)-5,6,7,8-tetrahydro-[1]-benzothieno-[2,3-d]-pyrimidin-2-yl)-phenyl]acetic acid, m.p. 214.

The examples below relate to pharmaceutical preparations.

### EXAMPLE A: INJECTION VIALS

A solution of 100 g of an active ingredient of the formula I and 5 g of disodium hydrogenphosphate in 3 l of bidistilled water is adjusted to pH 6.5 using 2N hydrochloric acid, sterile filtered, transferred into injection vials, lyophilised under sterile conditions and sealed under sterile conditions. Each injection vial contains 5 mg of active ingredient.

### EXAMPLE B: SUPPOSITORIES

A mixture of 20 g of an active ingredient of the formula I is melted with 100 g of soya lecithin and 1400 g of cocoa butter, poured into moulds and allowed to cool. Each suppository contains 20 mg of active ingredient.

### EXAMPLE C: SOLUTION

A solution is prepared from 1 g of an active ingredient of the formula I, 9.38 g of NaH₂PO₄.2H₂O, 28.48 g of Na₂HPO₄.12H₂O and 0.1 g of benzalkonium chloride in 940 ml of bidistilled water. The pH is adjusted to 6.8, and the solution is made up to 1 l and sterilised by irradiation. This solution can be used in the form of eye drops.

### EXAMPLE D: OINTMENT

500 mg of an active ingredient of the formula I are mixed with 99.5 g of Vaseline under aseptic conditions.

### EXAMPLE E: TABLETS

A mixture of 1 kg of an active ingredient of the formula I, 4 kg of lactose, 1.2 kg of potato starch, 0.2 kg of talc and 0.1 kg of magnesium stearate is pressed to give tablets in a conventional manner in such a way that each tablet contains 10 mg of active ingredient.

### EXAMPLE F: COATED TABLETS

Tablets are pressed analogously to Example E and subsequently coated in a conventional manner With a coating of sucrose, potato starch, talc, tragacanth and dye.

### EXAMPLE G: CAPSULES

2 kg of an active ingredient of the formula I are introduced into hard gelatine capsules in a conventional manner in such a way that each capsule contains 20 mg of the active ingredient.

### EXAMPLE H: AMPOULES

A solution of 1 kg of an active ingredient of the formula I in 60 l of bidistilled water is sterile filtered, transferred into ampoules, lyophilised under sterile conditions and sealed under sterile conditions. Each ampoule contains 10 mg of active ingredient. EXAMPLE I: INHALATION SPRAY

14 g of an active ingredient of the formula I are dissolved in 10 l of isotonic NaCl solution, and the solution is transferred into commercially available spray containers with a pump mechanism. The solution can be sprayed into the mouth or nose. One spray shot (about 0.1 ml) corresponds to a dose of about 0.14 mg.

1. Use of compounds of the formula I 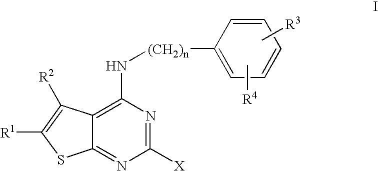
in which
R¹ and R² are each, independently of one another, H, A or Hal, where one of the radicals R¹ and R² is always ≠H,
R¹ and R² together are alternatively alkylene having 3-5 carbon atoms,
R³ and R⁴ are each, independently of one another, H, A, OA, OH or Hal,
R³ and R⁴ together are alternatively alkylene having 3-5 carbon atoms, —O—CH₂—CH₂—, —O—CH₂—O— or —O—CH₂—CH₂—O—,
X is R⁵ or R⁶ which is monosubstituted by R⁷,
R⁵ is linear or branched alkylene having 1-10 carbon atoms, in which one or two CH₂ groups may be replaced by —CH═CH— groups, or is —C₆H₄—(CH₂)ₘ—,
R⁶ is cycloalkylalkylene having 6-12 carbon atoms,
R⁷ is COOH, COOA, CONH₂, CONHA, CON(A)₂ or CN,
A is alkyl having from 1 to 6 carbon atoms,
Hal is F, Cl, Br or I ,
m is 1 or 2, and
n is 0, 1, 2 or 3,
and their physiologically acceptable salts and/or solvates, for the preparation of a medicament for the treatment of angina, high blood pressure, high pulmonary pressure, congestive heart failure, atherosclerosis, conditions of reduced patency of heart vessels, peripheral vascular diseases, strokes, bronchitis, allergic asthma, chronic asthma, allergic rhinitis, glaucoma, irritable bowel syndrome, tumours, renal insufficiency, liver cirrhosis and for the treatment of female sexual disorders.

2. Use of compounds of the formula I according to claim 1
(a) 3-[4-(3-chloro-4-methoxybenzylamino)-5,6,7,8-tetrahydro-[1]-benzothieno-[2,3-d]-pyrimidin-2-yl]propionic acid;
(b) 4-[4-(3,4-methylenedioxybenzylamino)-5,6,7,8-tetrahydro-[1]-benzothieno-[2,3-d]-pyrimidin-2-yl]butyric acid;
(c) 7-[4-(3,4-methylenedioxybenzylamino)-5,6,7,8-tetrahydro-[1]-benzothieno-[2,3-d]-pyrimidin-2-yl]heptanoic acid;
(d) 7-[4-(3-chloro-4-methoxybenzylamino)-5,6,7,8-tetrahydro-[1]-benzothieno-[2,3-d]-pyrimidin-2-yl]heptanoic acid;
(e) 5-[4-(3-chloro-4-methoxybenzylamino)-5,6,7,8-tetrahydro-[1]-benzothieno-[2,3-d]-pyrimidin-2-yl]valeric acid;
(f) 5-[4-(3-chloro-4-methoxybenzylamino)-6-methylthieno-[2,3-d]-pyrimidin-2-yl]valeric acid;
(g) 4-[4-(3-chloro-4-methoxybenzylamino)-6-methylthieno-[2, 3-d]-pyrimidin-2-yl]butyric acid;
(h) 4-[4-(3,4-methylenedioxybenzylamino)-6-methylthieno-[2, 3-d]-pyrimidin-2-yl]butyric acid;
(i) 2-{4-[4-(3-chloro-4-methoxybenzylamino)-5,6,7,8-tetrahydro-[1]-benzothieno-[2,3-d]-pyrimidin-2-yl]-cyclohexyl-1-yl}acetic acid;
(k) 5-[4-(3,4-methylenedioxybenzylamino)-6-methylthieno-[2,3-d]-pyrimidin-2-yl]valeric acid;
and their physiologically acceptable salts and/or solvates, for the preparation of a medicament for the treatment of angina, high blood pressure, high pulmonary pressure, congestive heart failure, atherosclerosis, conditions of reduced patency of heart vessels, peripheral vascular diseases, strokes, bronchitis, allergic asthma, chronic asthma, allergic rhinitis, glaucoma, irritable bowel syndrome, tumours, renal insufficiency, liver cirrhosis and for the treatment of female sexual disorders.

3. Use of
5-[4-(3-chloro-4-methoxybenzylamino)-5,6,7,8-tetrahydro-[1]-benzothieno-[2,3-d]-pyrimidin-2-yl]valeric acid
and its physiologically acceptable salts and/or solvates for the preparation of a medicament for the treatment of high pulmonary pressure.

4. Use of
5-[4-(3-chloro-4-methoxybenzylamino)-5,6,7,8-tetrahydro-[1]-benzothieno-[2,3-d]-pyrimidin-2-yl]valeric acid, ethanolamine salt,
for the preparation of a medicament for the treatment of high pulmonary pressure.

5. Use of
5-[4-(3-chloro-4-methoxybenzylamino)-5,6,7,8-tetrahydro-[1]-benzothieno-[2,3-d]-pyrimidin-2-yl]valeric acid, sodium salt,
for the preparation of a medicament for the treatment of high pulmonary pressure.
